(19) United States
(12) Patent Application Publication
(10) Pub. No.: US 2019/0322978 A1
(43) Pub. Date: Oct. 24, 2019

(54) ARTIFICIAL LEAF

(71) Applicant: Florida Atlantic University Board Of Trustees, Boca Raton, FL (US)

(72) Inventor: Vithulan Suthakaran, Boca Raton, FL (US)

(21) Appl. No.: 16/390,290

(22) Filed: Apr. 22, 2019

Related U.S. Application Data
(60) Provisional application No. 62/660,508, filed on Apr. 20, 2018.

(51) Int. Cl. C12M 1/00; B01D 53/84; C12N 1/12

(52) U.S. Cl. CPC C12M 43/04; C12M 21/02; B01D 2257/504; C12N 1/12; B01D 2251/95; B01D 53/84

(57) ABSTRACT

An apparatus for removing carbon dioxide from a gas mixture includes at least one artificial leaf. The artificial leaf includes a light transmissive, biodegradable and carbon dioxide and oxygen permeable hydrogel having embedded therein a photosynthetic cyanobacteria capable of the fixing carbon dioxide and a nutrition source for the cyanobacteria. A method of removing carbon dioxide from a gas and a method a making an apparatus for removing carbon dioxide from a gas are also disclosed.

### CROSS-REFERENCE TO RELATED APPLICATIONS

This application claims priority to U.S. Provisional Application No. 62/660,508 filed on Apr. 20, 2018, entitled “THE DEVELOPMENT OF AN ARTIFICIAL LEAF”, the entire disclosure of which is hereby incorporated herein by reference.

### FIELD OF THE INVENTION

The present invention carbon dioxide fixation, and more particularly on carbon dioxide fixation that utilizes solar radiation.

### BACKGROUND OF THE INVENTION

Every year, over 38 billion tons of carbon dioxide are released into the atmosphere due to various human related activities. With excess CO₂ production, many critical problems arise such as global warming, higher levels of air pollution, and ocean acidification. Recent studies state that excess amounts carbon dioxide becoming trapped within the earth's atmosphere is the number one leading cause for global warming. When unbeneficial amounts of greenhouse gases become trapped in the atmosphere, it creates a layer within earth preventing sunlight (which is absorbed through earth's surface and transformed into heat) from radiating back into space. This essentially causes large amounts of heat to become trapped within earth due to the different amounts of greenhouse gasses in the atmosphere. Due to this, glaciers are melting, severe droughts are occurring, sea levels are rising, and habitats are being disrupted. Although plants can filter large amounts of carbon dioxide, land, which may not always be available is required to complete this process. Current alternatives involve developing a transportable biomimetic artificial leaf to mimic the process of photosynthesis.

Biomimicry, the imitation of models, systems, and elements of nature for the purpose of solving complex human problems has been a common research strategy to develop biological solutions for global issues. Topics range from developing a hybrid material mimicking the barnacle's ability to cling to rocks to engineering a soft autonomous robot that moves via peristalsis. Within the biomimicry field, recent studies have brought attention to the idea of developing a fully biomimetic leaf which mimics the process of photosynthesis. Such device would utilize a chemical process to convert sunlight, water, and carbon dioxide into oxygen and carbohydrates, aiding in long term long term space exploration as well as a fully biomimetic device that produces and filters oxygen and carbon dioxide without the need for land. Current research on this subject is very limited, as it involves developing catalysts out of expensive and rare materials such as platinum, and researchers have reported low efficiencies in conducting artificial photosynthesis.

In natural photosynthesis, chlorophyll, a green pigment found in plants is an important factor in photosynthesis as it allows plants to absorb energy from a light source. Similar to chlorophyll, artificial systems use a molecule called a photosensitizer to absorb visible light. One molecular structure is tris(bipyridine)ruthenium(II) chloride, which is a compound that is used for visible light absorption due to its efficiency.

### SUMMARY OF THE INVENTION

An apparatus for removing carbon dioxide from a gas mixture includes at least one artificial leaf. The artificial leaf can include a light transmissive, biodegradable and carbon dioxide and oxygen permeable hydrogel having embedded therein a photosynthetic cyanobacteria capable of fixing carbon dioxide and a nutrition source for the cyanobacteria. The gas can be air.

The cyanobacteria can include Spirulina. The nutrition source can include Zarrouk medium and poloxamer such as Poloxamer 407. The hydrogel can include a cyto-compatible calcium alginate hydrogel.

The artificial leaf can be planar. The artificial leaves can have a thickness that is less than the length or the width of the artificial leaf. The apparatus can include a plurality of artificial leaves and a support structure. The support structure can hold the artificial leaves in spaced relation to one another. The support structure can also position the artificial leaves for exposure to solar radiation. The support structure can be configured to resemble a tree, and the artificial leaves can be configured to resemble leaves of differing designs.

A method of removing carbon dioxide from a gas includes the step of providing at least one artificial leaf. The artificial leaf can include a light transmissive, biodegradable and carbon dioxide and oxygen permeable hydrogel having embedded therein a photosynthetic cyanobacteria capable of fixing carbon dioxide and a nutrition source for the cyanobacteria. The artificial leaf can be positioned so as to receive light radiation. The gas can be air, and the light radiation can be solar radiation.

The method can include the step of, after a period of exposure to solar radiation, placing the artificial leaf in the ground as a plant nutrient. The method can include the step of positioning the artificial leaf so as to be contacted by an effluent gas stream from a carbon dioxide generating process.

A method of making an apparatus for removing carbon dioxide from the atmosphere can include the step of mixing hydrogel precursor compounds with water, cyanobacteria and a nutrient composition for the cyanobacteria. The mixture is placed in a mold and the hydrogel precursor compounds are gelled into a hydrogel, forming a light transmissive, carbon dioxide and oxygen permeable hydrogel to retain the cyanobacteria within the hydrogel and form a artificial leaf. The artificial leaf can be removed from the mold.

### BRIEF DESCRIPTION OF THE DRAWINGS

There are shown in the drawings embodiments that are presently preferred it being understood that the invention is not limited to the arrangements and instrumentalities shown, wherein:

FIG. 1 is a schematic diagram of an apparatus for fixing carbon from carbon dioxide gas.

FIG. 2 is a schematic diagram of an alternative embodiment for indoor usage.

FIG. 3 is a schematic diagram of an alternative embodiment for removing carbon dioxide from process gas streams.

### DETAILED DESCRIPTION OF THE INVENTION

An apparatus for removing carbon dioxide from a gas mixture includes at least one artificial leaf. The artificial leaf includes a light transmissive, biodegradable and carbon dioxide and oxygen permeable hydrogel. The hydrogel has embedded therein a photosynthetic cyanobacteria capable of the fixing carbon from carbon dioxide, and also a nutrition source for the cyanobacteria.

Hydrogels are highly absorbent water-based materials which are gel type solids. Since hydrogels are mainly water based, they act as a beneficial resource in encapsulating photosynthesizing cyanobacteria. The hydrogel scaffold for the cyanobacteria should be permeable to carbon dioxide and oxygen, light transmissive, and non-toxic to the cyanobacteria to sustain the cyanobacteria and to allow the cyanobacteria to remove carbon dioxide by photosynthesis.

The hydrogel can be made from different materials. One such hydrogel comprises a calcium alginate hydrogel. Sodium alginate as a starting material is an environmentally friendly and biodegradable material which polymerizes from a liquid solution into the hydrogel when reacted with calcium chloride (CaCl₂)). The calcium alginate hydrogel so formed is nontoxic to the cyanobacteria and to plants and the environment. It is also permeable to carbon dioxide and oxygen, and is light transmissive.

The hydrogel can further include poloxamer 407. Poloxamer 407 is a cell culturing bio reagent which has been determined as a viable tool in cell encapsulation. Poloxamer 407 has the general formula:

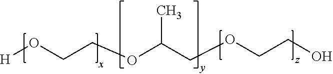

The cyanobacteria that are suitable for the invention are capable of fixing carbon and have acceptable sustainability in the hydrogel. One such cyanobacteria comprises Spirulina. Other cyanobacteria are possible. Cyanobacteria is a type of bacteria who receive energy through the process of photosynthesis. These organisms are found in most aquatic habitats. A unique feature that they possess is that these prokaryotes have both nitrogen fixation and carbon fixation properties. Overall, they are the only prokaryote able to both produce oxygen and filter carbon dioxide. However, when the aquatic habitat is warm, and the water is slow moving, harmful cyanobacterial proliferations can occur. Cyanobacterial blooms pose a major threat to water ecosystems, as the cause for both plants and fish to die, due to the high number of algae in the water. As cyanobacteria blooms occur, the surface of the water is covered in cyanobacteria, preventing sunlight from reaching the lower ecosystems. This lack of sunlight is extremely harmful as it can damage different types of ecosystems as well as alter the temperature of the water. Due to this, encapsulating the cyanobacteria in a hydrogel restricts the movement of the cyanobacteria into aquatic environments.

The light radiation can be solar radiation. The light radiation source can also be artificial light generated by one or more artificial light generating apparatus. If artificial, the light should include wavelengths that are suitable for the cyanobacteria that is used.

The invention provides a biomimetic device to mimic the process of photosynthesis in order to filter large amounts of carbon dioxide from the atmosphere, while producing oxygen. The artificial leaf incorporates photosynthesizing cyanobacteria into a cyto-compatible hydrogel, due to the algae's robustness/ability to survive in variable conditions. Since hydrogels contain over 90% water, cyanobacteria are able to sustain their structures in a cyto-compatible hydrogel. The hydrogel is porous such that carbon dioxide can reach the cyanobacteria, and oxygen generated by the cyanobacteria can escape.

The artificial leaf is capable of taking a variety of sizes and shapes. In one aspect, the artificial leaf is planar, and the thickness can be less than the length or the width. The shape and size of the artificial leaf can be adjusted to resemble a leaf for aesthetic purposes.

A plurality of artificial leaves and a support structure can be provided. The support structure holds the leaves in spaced relation to one another. Each artificial leaf can be supported in such a position as to provide exposure to solar radiation and to provide contact with the gas. The support structure can be configured to resemble a tree, and the artificial leaves can be configured to resemble leaves.

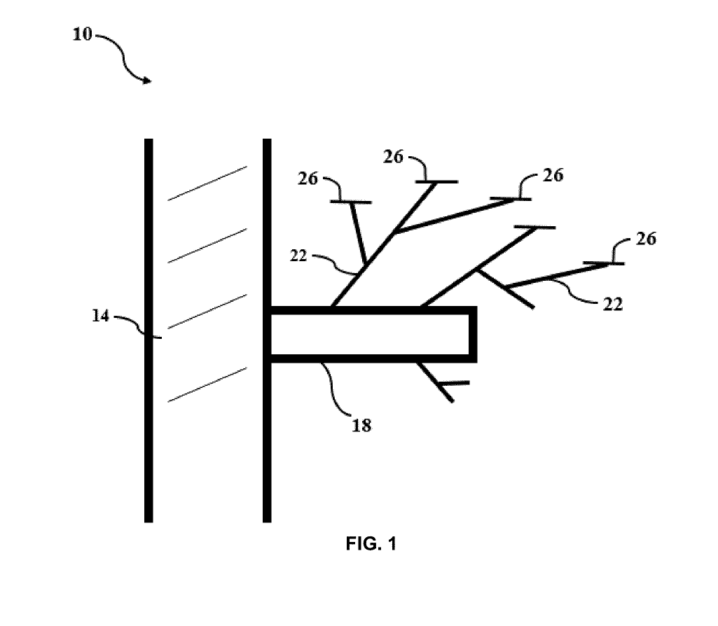

There is shown in FIG. 1 an apparatus 10 for removing carbon dioxide from a gas mixture. The apparatus is generally configured to resemble a plant or tree, in either realistic or abstract form. A central support or trunk 14 can have one or more branches 18 and can have smaller branches 22 extending from the branches 18. Artificial leaves 26 generally in the shape of leaves are positioned by the supporting structure so as to receive solar radiation, and also a flow of air or other gas mixture from which to receive carbon dioxide. Other designs are possible.

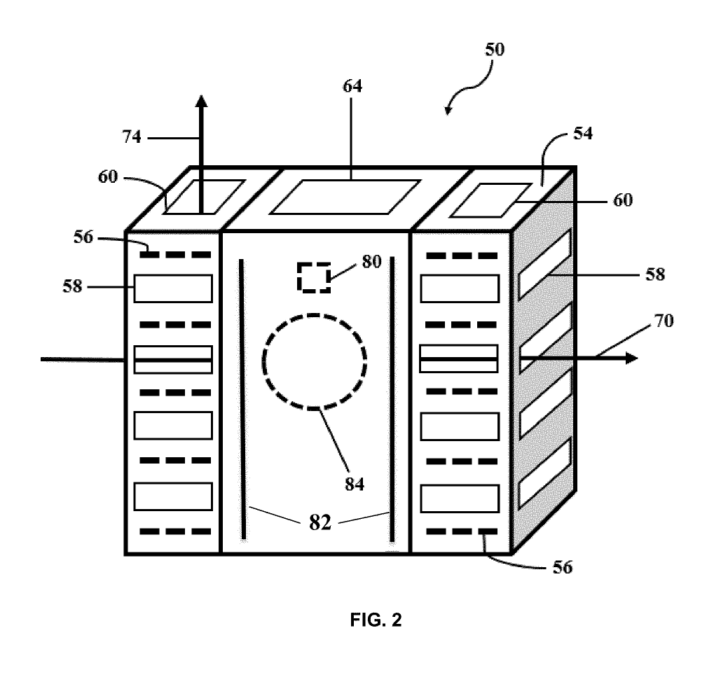

The artificial leaves can remove carbon dioxide from a number of gas streams. The gas can be air. The apparatus can remove carbon dioxide from outdoor air sources, or indoor air sources. There is shown schematically in FIG. 2 an apparatus 50 that is suitable for removing carbon dioxide from an indoor air source. The apparatus 50 has a housing 54 in which are secured a plurality of artificial leaves 56. Any number of artificial leaves 56 can be provided depending on the dimensions of the housing 54.

The housing 54 can have a plurality of ventilation openings 58 to permit air or other gas mixtures to circulate through the housing 54 as indicated by arrow 70. Openings 60 at an upper surface of the housing 54 can be provided to permit ambient light to enter the housing 54. Alternatively, one or more artificial light sources 82 can be mounted to or within or in proximity to the housing 54. The openings 60 can also permit ventilation as indicated by arrow 74. Alternatively, the opening 60 can be closed by a light transmissive material such as plastic or glass. A carbon dioxide sensor 80 can be provided to sense the carbon dioxide levels and can provide a signal to a processor (not shown). The sensed carbon dioxide levels can be utilized to determine when the artificial leaves 56 have ceased to function at an acceptable level and should be replaced. A fan or blower 84 can be provided to assist with the ventilation of air through the unit. Other designs are possible.

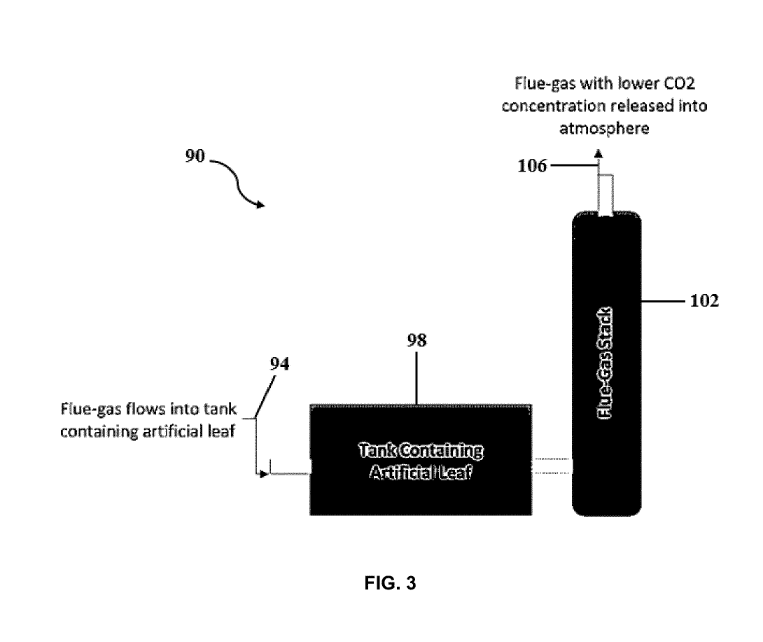

The apparatus can also be used to remove carbon dioxide from non-atmospheric, industrial gas streams, for example from flue gas streams. The gas streams must not contain components that are toxic to the cyanobacteria. A schematic depiction of such a process gas stream apparatus 90 is shown in FIG. 3. The process gas stream 94 enters a housing 98 which contains a plurality of artificial leaves according to the invention. The apparatus can receive solar radiation or artificial light radiation. The cyanobacteria within the artificial leaves removes carbon dioxide from the gas stream. The cleansed gas stream with reduced carbon dioxide content enters a flue-gas stack 102 and exhausts at 106. Other designs are possible.

The Spirulina cyanobacteria used to develop the artificial leaves is one of the only type of cyanobacteria that are not atmospheric nitrogen fixing cyanobacteria. These cyanobacteria therefore do not provide nitrogen to promote plant growth. The calcium chloride that is used to form the calcium alginate hydrogel, however, is typically in excess or can be added in such quantities as to provide an excess of calcium chloride. Calcium chloride is commonly used as a fertilizer for plants, so the calcium chloride in the artificial leaf will promote plant growth. Also, the expired Spirulina are a known nutritional source for some plants. The artificial leaves therefore when no longer useful to remove carbon dioxide gas and generate oxygen can be replaced, and the spent leaves used for plant nutrition by either placing directly into the soil or ground into pieces and placed into the soil similar to fertilizer. The method of the invention can therefore further include the step of, after a period of exposure to solar radiation, placing the artificial leaf in the ground as a plant nutrient. It is also possible to incorporate carbon fixing cyanobacteria with cyanobacteria that fix nitrogen, such that the spent artificial leaves provide a more complete nutritional source for plants that includes nitrogen.

The nutrition source can vary based on the bacteria that are used and the conditions in which the cyanobacteria will be placed such as temperature, gas composition and the like. One such nutritional source for cyanobacteria is Zarrouk medium. Zarrouk medium has the composition:

NaHCO₃=16.8 g/L

K₂HPO₄=0.5 g/L

NaNO₃=2.5 g/L

MgSO₄*7H₂O=0.2 g/L

CaCl₂)=0.04 g/L

FeSO₄*7H₂O=0.01 g/L

EDTA=0.08 g/L

Solution A5=1 mL

Solution B6=1 mL

A method of making an apparatus for removing carbon dioxide from a gas can include the steps of mixing hydrogel precursor compounds with water, cyanobacteria and a nutrient composition for the cyanobacteria. The mixture is placed in a mold and forms a light transmissive hydrogel to retain the cyanobacteria within the hydrogel and form a artificial leaf. The artificial leaf is then removed from the mold.

### Example

Sodium alginate aqueous solution development: Cyanobacteria was obtained from the Carolina Biological Supply company and was kept sealed in a BSL-2 laboratory to avoid any contamination. Using an analytical balance, 5 grams of sodium alginate was measured and mixed in a beaker containing 100 ml of deionized water to develop a 5% solution of sodium alginate. After the sodium alginate was dissolved in the deionized water, the solution was autoclaved at 121° C. for 20 minutes to sterilize the solution. The beaker was removed from the autoclave and the solution was placed on a hotplate preheated to 40° C. The beaker was allowed to remain on the hotplate for 15 to minutes to adjust to the temperature.

Cyto-compatible sodium alginate solution development: After the aqueous solution of sodium alginate was cooled to around 40° C., 20 ml of cyanobacteria was mixed into the solution. A 2% solution of poloxamer 407 was developed by measuring 2 grams of poloxamer 407 and mixing it with 100 ml of deionized water. 10 ml of the 2% poloxamer 407 solution was poured into the beaker containing sodium alginate and cyanobacteria. This solution was mixed thoroughly.

Cyto-compatible calcium alginate hydrogel development: Two microscope plates and parafilm was utilized to create a rectangular mold to hold the aqueous sodium solution in order to develop the cyto-compatible calcium alginate hydrogel. The cyto-compatible sodium alginate solution was micropipetted into the rectangular molds. Each mold was placed into a container and was fully submerged in a 2% solution of calcium chloride for a couple of hours to polymerize the cyto-compatible sodium alginate solution into an artificial leaf, essentially developing the artificial leaf. After 3-5 hours, the molds were taken out of the calcium chloride and carefully removed to obtain the finished artificial leaves.

Data was collected by placing the artificial leaves in a sealed tank for 8 minutes. Using a carbon dioxide tank, 5000 ppm of carbon dioxide was released into the testing apparatus using a CO₂ tank and the CO₂ was measured using a Pasco carbon dioxide gas sensor. Testing was performed by measuring how much carbon dioxide was filtered from the tank, and oxygen was produced over a timed interval. Prior to data collection, controlled tests were run to demonstrate that the tank was adequately sealed.

Data collection involved constructing a sealed tank to determine the amount of carbon dioxide the artificial leaves could filter, as well as the amount of oxygen that could be produced. Prior to data collection, control tests were run by adding 5,000 ppm of carbon dioxide in the tank while no units were present. Results showed that there was no decrease in the CO₂ ppm, establishing that the testing apparatus was adequately sealed. The artificial leaves were placed in the sealed tank and 5,000 ppm of carbon dioxide was added into the testing environment. After the carbon dioxide was added into the tank, an artificial light source was utilized to aid the process of photosynthesis. For both the oxygen production rate and carbon dioxide filtration rate, data sets were run at an 8-minute interval five different times and were measured using a Pasco carbon dioxide and oxygen gas sensor. For the carbon dioxide tests in eight minutes, 96% of the CO₂ was filtered in trial 1, 98% in trial 2, 96% in trial 3, 97% in trial 4, and 95% in trial 5. For the oxygen tests in eight minutes, the oxygen increased 2800 ppm in trial 1, 3100 ppm in trial 2, 3000 ppm in trial 3, 3150 ppm in trial 4, and 3000 in trial 5.

Using a spectrophotometer, it was determined that the artificial leaf and aqueous solution prior to gelling were growing cyanobacteria at similar rates for three hours over a six-hour interval. However, after the third hour, the cyanobacteria were growing much more efficiently in the hydrogel of the artificial leaf rather than the aqueous solution. Tests were run for five more days, and results again showed that the artificial leaf grew cyanobacteria at a faster rate. The artificial leaf creates a much more sustainable environment for cyanobacteria because the calcium chloride, which is not present in the aqueous solution, could be acting as a possible fertilizer for the bacteria.

Data Summary: Carbon Dioxide Filtration Test. It was determined that when 5,000 ppm of carbon dioxide was released into a sealed tank, the artificial leaf was able to filter 98% of the carbon dioxide within eight minutes. Oxygen Production Test. It was determined that when the artificial leaves were place in a sealed tank for eight minutes, they were able to produce 3000 ppm of oxygen. Biomass Production. It was determined that the artificial leaves created a much more sustainable environment to have cyanobacteria grow, rather than an aqueous solution.

The carbon fixation apparatus shown in the drawings and described in detail herein disclose arrangements of elements of particular construction and configuration for illustrating preferred embodiments of structure and method of operation of the present invention. It is to be understood however, that elements of different construction and configuration and other arrangements thereof, other than those illustrated and described may be employed in accordance with the spirit of the invention, and such changes, alternations and modifications as would occur to those skilled in the art are considered to be within the scope of this invention as broadly defined in the appended claims. In addition, it is to be understood that the phraseology and terminology employed herein are for the purpose of description and should not be regarded as limiting.

With respect to the above description then, it is to be realized that the optimum dimensional relationships for the parts of the invention, to include variations in size, materials, shape, form, function and manner of operation, assembly and use, would be apparent to one skilled in the art, and all equivalent relationships to those illustrated in the drawings and described in the specification are intended to be encompassed by the present invention. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. An apparatus for removing carbon dioxide from a gas mixture, comprising at least one artificial leaf, the artificial leaf comprising a light transmissive, biodegradable and carbon dioxide and oxygen permeable hydrogel having embedded therein a photosynthetic cyanobacteria capable of the fixing carbon dioxide and a nutrition source for the cyanobacteria.

2. The apparatus of claim 1, wherein the cyanobacteria comprises Spirulina.

3. The apparatus of claim 1, wherein the nutrition source comprises Zarrouk medium and Poloxamer 407.

4. The apparatus of claim 1, wherein the hydrogel comprises a cyto-compatible calcium alginate hydrogel.

5. The apparatus of claim 1, further comprising a poloxamer.

6. The apparatus of claim 5, wherein the poloxamer is Poloxamer 407.

7. The apparatus of claim 1, wherein the artificial leaf is planar.

8. The apparatus of claim 1, comprising a plurality of artificial leaves and a support structure, wherein the support structure holds the artificial leaves in spaced relation to one another.

9. The apparatus of claim 8, wherein the support structure positions the artificial leaves for exposure to solar radiation.

10. The apparatus of claim 8, wherein the support structure is configured to resemble a tree, and the artificial leaves are configured to resemble leaves.

11. The apparatus of claim 1, wherein the artificial leaves have a thickness that is less than the length or the width of the artificial leaf.

12. The apparatus of claim 1, wherein the gas is air.

13. A method of removing carbon dioxide from a gas, comprising the steps of:
providing at least one artificial leaf, the artificial leaf comprising a light transmissive, biodegradable and carbon dioxide and oxygen permeable hydrogel having embedded therein a photosynthetic cyanobacteria capable of fixing carbon dioxide and a nutrition source for the cyanobacteria; and
positioning the artificial leaf so as to receive light radiation.

14. The method of claim 13, wherein the cyanobacteria comprises Spirulina.

15. The method of claim 13, wherein the nutrition source comprises Zarrouk medium and Poloxamer 407.

16. The method of claim 13, wherein the hydrogel comprises a cyto-compatible calcium alginate hydrogel.

17. The method of claim 13, further comprising a poloxamer.

18. The method of claim 17, wherein the poloxamer is Poloxamer 407.

19. The method of claim 13, wherein the artificial leaf is planar.

20. The method of claim 13, comprising a plurality of artificial leaves and a support structure, wherein the support structure holds the artificial leaves in spaced relation to one another.

21. The apparatus of claim 13, wherein each artificial leaf is positioned for exposure to solar radiation.

22. The method of claim 13, wherein the support structure is configured to resemble a tree, and the artificial leaves are configured to resemble leaves.

23. The method of claim 13, wherein the artificial leaves have a thickness that is less than the length or the width of the artificial leaf.

24. The method of claim 13, further comprising the step of after a period of exposure to solar radiation, using the artificial leaf as a plant nutrient.

25. The method of claim 13, wherein the artificial leaf is positioned to be contacted by an effluent gas stream from a carbon dioxide generating process.

26. The method of claim 13, wherein the gas is air.

27. The method of claim 13, wherein the light radiation is solar radiation.

28. A method of making an apparatus for removing carbon dioxide from the atmosphere, comprising the steps of:
mixing hydrogel precursor compounds with water, cyanobacteria and a nutrient composition for the cyanobacteria;
placing the mixture in a mold and gelling the hydrogel precursor compounds into a hydrogel, forming a light transmissive, carbon dioxide and oxygen permeable hydrogel to retain the cyanobacteria within the hydrogel and form a artificial leaf; and
removing the artificial leaf from the mold.
